(19) United States
(12) Patent Application Publication
(10) Pub. No.: US 2021/0030537 A1
(43) Pub. Date: Feb. 4, 2021

(54) Tethering System For A Prosthetic Heart Valve

(71) Applicant: Tendyne Holdings, Inc., St. Paul, MN (US)

(72) Inventor: Zachary J. Tegels, Minneapolis, MN (US)

(21) Appl. No.: 16/943,431

(22) Filed: Jul. 30, 2020

Related U.S. Application Data
(60) Provisional application No. 62/880,839, filed on Jul. 31, 2019.

(51) Int. Cl. A61F 2/24

(52) U.S. Cl. CPC A61F 2/2418; A61F 2/2409; A61F 2250/0097; A61F 2250/0075; A61F 2220/0016

(57) ABSTRACT

According to one aspect of the disclosure, a system may include a surgical prosthetic heart valve having a frame, prosthetic leaflets within the frame configured to allow blood to flow through the frame in an antegrade direction but to substantially block blood from flowing through the frame in a retrograde direction, and a cuff configured to be sutured to a native valve annulus of a heart of a patient. The system may include a valve tether having a first end configured to be coupled to the surgical prosthetic heart valve, and an anchor configured to be secured to a ventricular apex of the heart of the patient. The valve tether may include a second end opposite the first end, the second end configured to be operably coupled to the anchor.

### CROSS-REFERENCE TO RELATED APPLICATIONS

The present application claims the benefit of the filing date of U.S. Provisional Patent Application No. 62/880,839 filed Jul. 31, 2019, the disclosure of which is hereby incorporated herein by reference.

### BACKGROUND OF THE DISCLOSURE

The present disclosure relates to devices and methods for attaching a tether to a prosthetic heart valve.

Prosthetic heart valves typically fall into one of two general categories: collapsible prosthetic heart valves or surgical prosthetic heart valves. Prosthetic heart valves that are collapsible to a relatively small circumferential size can be delivered into a patient less invasively than valves that are not collapsible. For example, a collapsible valve may be delivered into a patient via a tube-like delivery apparatus such as a catheter, a trocar, a laparoscopic instrument, or the like. This collapsibility can avoid the need for a more invasive procedure such as full open-chest, open-heart surgery. Surgical prosthetic heart valves, on the other hand, typically are not collapsible and may require full open-chest, open-heart surgery.

Surgical heart valves are typically attached to a patient's native anatomy, for example at a native valve annulus, via suturing. This suture attachment is typically the primary mode of securing a surgical heart valve in a desired position within the anatomy. Problems may arise with a surgical heart valve if the sutures become loose, degrade, or otherwise become damaged.

Thus, it would be preferable to provide additional mechanisms of securing a surgical heart valve during implantation to help reduce the likelihood of the surgical heart valve becoming inadequately supported in the anatomy, or to otherwise provide additional mechanisms of supporting a surgical valve after an initial implantation to mitigate potential problems arising from a failure of the primary mode of securement.

### BRIEF SUMMARY

According to one embodiment of the disclosure, a system includes a surgical prosthetic heart valve, a valve tether, and an anchor. The surgical prosthetic heart valve may have a frame, prosthetic leaflets within the frame configured to allow blood to flow through the frame in an antegrade direction but to substantially block blood from flowing through the frame in a retrograde direction, and a cuff configured to be sutured to a native valve annulus of a heart of a patient. The valve tether may have a first end configured to be coupled to the surgical prosthetic heart valve. The anchor may be configured to be secured to a ventricular apex of the heart of the patient. The valve tether may include a second end opposite the first end, the second end configured to be operably coupled to the anchor.

According to a further embodiment of the disclosure, a system for tethering a prosthetic heart valve to a heart of a patient may include a valve tether and an anchor. The valve tether may have a first end configured to be coupled to the prosthetic heart valve. The anchor may be configured to be secured to a ventricular apex of the heart of the patient. The valve tether may be formed of a rigid material and may include a securing mechanism on the first end to secure the first end of the valve tether to the prosthetic heart valve. The valve tether may include a second end opposite the first end configured to be operably coupled to the anchor.

According to yet another embodiment of the disclosure is a method for tethering a prosthetic heart valve to a heart of a patient during a second interventional procedure, the prosthetic heart valve having already been implanted into the patient during an earlier first interventional procedure. The method may include inserting the first end of a valve tether into an incision on a ventricular wall of the heart of the patient. The first end of the valve tether may be advanced to the previously implanted prosthetic heart valve. The first end of the valve tether may be attached to the prosthetic heart valve. A second end of the valve tether opposite the first end may be operably attached to an anchor. The anchor may be secured to the ventricular wall of the heart of the patient.

As used herein, the term “prosthetic heart valve” should be understood to encompass both surgical prosthetic heart valves and collapsible prosthetic heart valves, unless context clearly dictates otherwise.

### BRIEF DESCRIPTION OF THE DRAWINGS

FIG. 1 is a highly schematic cutaway representation of a human heart showing various delivery approaches.

FIG. 2 is a highly schematic representation of a native mitral valve and associated cardiac structures.

FIG. 3 is a simplified perspective view of a component of an illustrative embodiment of a surgical prosthetic heart valve.

FIG. 4 is a simplified elevational view of another component of a surgical prosthetic heart valve.

FIG. 5 is a simplified perspective view of an assembly of the components from FIGS. 3-4.

FIG. 6 is a front view of a tethering system according to one embodiment of the present invention.

FIG. 7 is a top view of a template according to one embodiment of the present invention.

FIG. 8 is a flowchart of a method of using the tethering system according to one embodiment of the present invention.

FIG. 9 is a front view of a tethering system according to another embodiment of the present invention.

FIG. 10A is a front view of a tethering system with a valve tether having a knotted securing mechanism according to another embodiment of the present invention.

FIG. 10B is a front view of a tethering system with a valve tether having a threaded securing mechanism according to another embodiment of the present invention.

FIG. 10C is a front view of a tethering system with a valve tether having a barbed securing mechanism according to another embodiment of the present invention.

### DETAILED DESCRIPTION

As used herein, the words “proximal” and “distal,” when used in connection with a medical device, refer to a position closer to and farther away from, respectively, an operator of the medical device. The term “inflow end,” when used in conjunction with a prosthetic heart valve, refers to the end of the prosthetic heart valve through which blood first flows when the prosthetic heart valve is operating normally, while the term “outflow end,” when used in conjunction with a prosthetic heart valve, refers to the end of the prosthetic heart valve through which bloods exits when the prosthetic heart valve is operating normally.

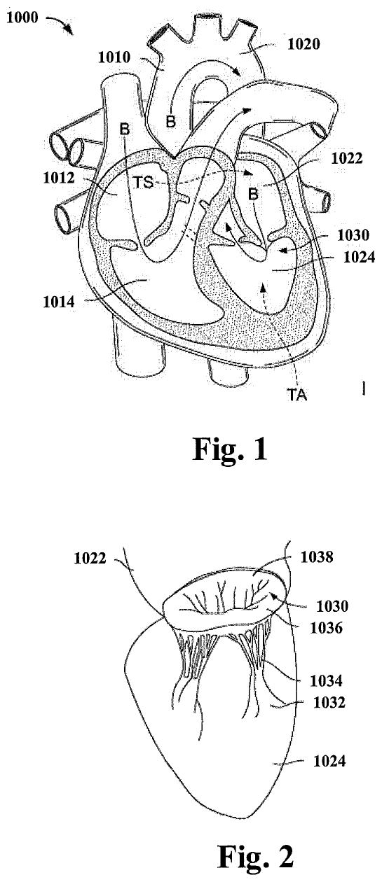

FIG. 1 is a highly schematic cutaway representation of human heart 1000. The human heart includes two atria and two ventricles: right atrium 1012 and left atrium 1022, and right ventricle 1014 and left ventricle 1024. Heart 1000 further includes aorta 1010 and aortic arch 1020. Disposed between left atrium 1022 and left ventricle 1024 is mitral valve 1030. Mitral valve 1030, also known as the bicuspid valve or left atrioventricular valve, is a dual-flap valve that opens as a result of increased pressure in left atrium 1022 as it fills with blood. As atrial pressure increases above that of left ventricle 1024, mitral valve 1030 opens and blood passes into left ventricle 1024. Blood flows through heart 1000 in the direction shown by arrows “B”.

A dashed arrow, labeled “TA,” indicates a transapical approach of implanting a prosthetic heart valve, in this case to replace mitral valve 1030. In transapical delivery, a small incision is made between the ribs and into the apex of left ventricle 1024 to deliver the prosthetic heart valve to the target site. A second dashed arrow, labeled “TS,” indicates a transseptal approach of implanting a prosthetic heart valve in which the valve is passed through the septum between right atrium 1012 and left atrium 1022. Transapical approach TA and transseptal approach TS are typically used in conjunction with the implantation of collapsible prosthetic heart valves, as opposed to surgical heart valves.

FIG. 2 is a more detailed schematic representation of native mitral valve 1030 and its associated structures. As previously noted, mitral valve 1030 includes two flaps or leaflets, posterior leaflet 1036 and anterior leaflet 1038, disposed between left atrium 1022 and left ventricle 1024. Cord-like tendons, known as chordae tendineae 1034, connect the two leaflets 1036, 1038 to the medial and lateral papillary muscles 1032. During atrial systole, blood flows from higher pressure in left atrium 1022 to lower pressure in left ventricle 1024. When left ventricle 1024 contracts in ventricular systole, the increased blood pressure in the chamber pushes leaflets 1036, 1038 to close, preventing the backflow of blood into left atrium 1022. Since the blood pressure in left atrium 1022 is much lower than that in left ventricle 1024, leaflets 1036, 1038 attempt to evert to the low pressure regions. Chordae tendineae 1034 prevent the eversion by becoming tense, thus pulling on leaflets 1036, 1038 and holding them in the closed position.

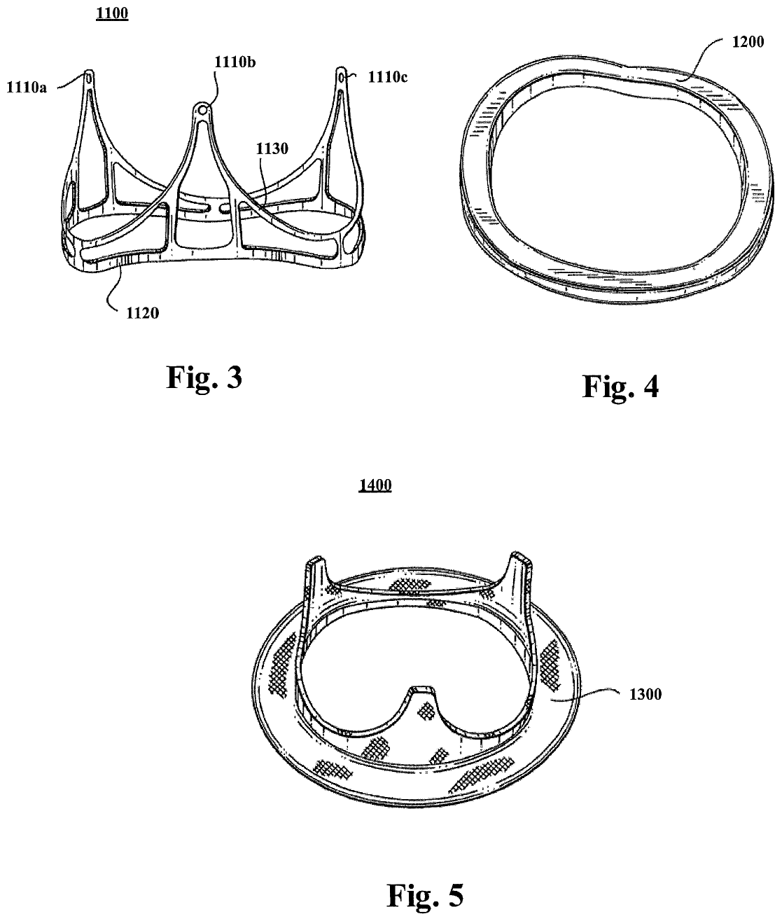

FIGS. 3-5 depict the components of an exemplary surgical prosthetic heart valve capable of being used both in the mitral and tricuspid valves, as disclosed in U.S. Pat. No. 8,353,954, the disclosure of which is hereby incorporated by reference herein. An illustrative embodiment of a first component 1100 of a surgical prosthetic heart valve is shown in FIG. 3. Component 1100 is a hollow, annular, stent-like structure (sometimes referred to for convenience herein simply as a stent). Stent 1100 is referred to as “hollow” because the interior that is bounded by its annular structure is open. Stent 1100 is typically made of metal such as titanium (e.g., Ti 6Al-4V ELI Grade 5). A typical technique for making stent 1100 is to cut it from a tube using a laser. Stent 1100 is then typically electro-polished.

Stent 1100 has three commissure portions or regions 1110a, 1110b, and 1110c that are equally spaced from one another around the circumference of the stent. Each commissure portion 1110a-c stands up from the annularly continuous base portion of stent 1100. The base portion includes a lower-most, inflow edge portion 1120. Inflow edge portion 1120 may be scalloped as one proceeds around stent 1100 to approximately match the natural scallop of the native valve annulus. In particular, this scallop rises in the vicinity of each commissure region 1110a-c, and it falls between each annularly adjacent pair of commissures. It should be understood that stent 1100 includes three commissure regions 1100a-c to accommodate three prosthetic heart valve leaflets, although in other embodiments, the stent may include more or fewer commissure regions, for example to accommodate a two-leaflet valve or a four-leaflet valve. Further, it should be understood that stent 1100 is adapted for use in a surgical prosthetic heart valve intended to replace a native aortic valve, although the principles discussed in connection with stent 1100 and the related surgical prosthetic heart valve may generally apply to surgical prosthetic heart valves intended to replace other native valves, such as native mitral valve 1030.

Inflow edge 1120, outflow edge 1130, and the flexibility of stent 1100 are designed to help ensure proper opening and coaptation of the finished surgical prosthetic heart valve in use. (Coaptation is the coming together of the outflow portions of the valve leaflets when the valve is closed.) Stent 1100 is further designed to decrease maximum stresses in use, which gives the finished valve an increased safety factor.

Although titanium is mentioned above as a typical material from which stent 1100 can be made, other materials are also possible. Some examples of other materials that may be suitable for use in making stent 1100 include Elgiloy MP35N, or polymers such as PEEK or acetal.

FIGS. 4-5 illustrate further possible components and steps in the manufacture of the illustrative embodiment of the surgical prosthetic heart valve being described. FIG. 4 shows an illustrative embodiment of a silicone cuff filler ring 1200; and FIG. 5 shows an assembly 1400 that includes stent 1100 and silicone cuff filler ring 1200 covered inside and out by fabric tube 1300. For example, stent 1100 and ring 1200 may be placed coaxially around the outside of a lower portion of fabric tube 1300. Ring 1200 may be located outside inflow edge portion 1120. The upper portion of sleeve 1300 may then be pulled down over the outside of components 1100 and 1200 and pulled tightly enough to conform to outflow edge portion 1130 as shown in FIG. 5. Sutures may be used to hold the above-described components together in the condition shown in FIG. 5. In particular, all of components 1100 and 1200 are completely covered inside and out by fabric 1300. Ring 1200 is located adjacent inflow edge portion 1120 and follows the scalloping of the inflow edge portion all the way around assembly 1400. The upper portion of fabric 1300 conforms closely to stent 1100 above ring 1200, and in particular, the upper portion of the fabric follows the scalloped outflow edge portion 1130 all the way around assembly 1400. It should be understood that prosthetic heart valve leaflets (which may be capable of being used in both surgical and collapsible prosthetic heart valves) are not shown in FIG. 5, but three prosthetic leaflets would be attached to assembly 1400, with adjacent pairs of leaflets being coupled to corresponding commissure regions 1110a-c to form a functioning surgical prosthetic heart valve.

A prosthetic heart valve may be implanted when a heart valve is not functioning correctly. For example, during ventricular systole, the mitral valve leaflets 1036, 1038 may not be able to close properly due to misaligned valve leaflets or weakened chordae tendineae 1034. Since the valve leaflets 1036, 1038 are unable to properly close, there may be backflow of blood from left ventricle 1024 to left atrium 1022. In such an instance, a prosthetic heart valve, including the surgical prosthetic heart valve described above, may be implanted within native mitral valve 1030 by attaching ring 1200 and/or surrounding fabric 1300 of the prosthetic heart valve to the annulus of the native mitral valve with the commissure portions 1110a-c pointing in an outflow direction, for example via suturing. Thus, during atrial systole, blood may flow from the higher pressure in left atrium 1022 to the lower pressure in left ventricle 1024 through the prosthetic leaflets of the prosthetic heart valve. During ventricular systole, the increased pressure from left ventricle 1024 will properly push the prosthetic leaflets closed, thereby preventing backflow into left atrium 1022.

However, in order for the prosthetic heart valve to properly function, the prosthetic heart valve must be appropriately secured to the native tissue. In a surgical prosthetic heart valve, ring 1200 is typically sutured to the native valve annulus to provide the requisite securement. In the case of a prosthetic mitral valve, the prosthesis must be attached such that, during ventricular systole, the attachment at ring 1200 is capable of withstanding the pressure generated by the contraction of left ventricle 1024 without displacement of the prosthesis.

Although the typical mode of attachment of a surgical prosthetic heart valve to a native valve annulus via suturing to the ring 1200 may be sufficient in certain cases, providing a secondary mode of attachment may provide benefits. For example, a tethering system may be provided to attach ring 1200 and/or stent 1100 to a structure of the heart, such as to the apex of left ventricle 1024 via an anchor positioned on the outside of the surface of the heart. Such a tethering system may assist in reducing the amount of stress placed on the primary attachment of ring 1200 during ventricular systole in a manner similar to chordae tendineae 1034. As described in greater detail below, the tethering system may include securing a first end of at least one tether to the ring 1200 and/or stent 1100 of the surgical prosthetic heart valve, and a second end of the at least one tether to an anchor on an epicardial surface of left ventricle 1024. As further described in greater detail below, the tethering system may be implemented during the initial surgery that implants the surgical prosthetic heart valve, or during a later surgery after the surgical prosthetic heart valve has already been implanted. In addition to providing additional securement of the surgical prosthetic heart valve to reduce the stress on the primary mode of attachment, the tension provided by the tethering system between the surgical prosthetic heart valve and the ventricular apex may separately enhance functioning of the heart. For instance, where the heart has deformed through distention or enlargement, the tethering system may function to support the wall of the ventricle by relieving some of the distention, thereby making it easier for the left ventricle to contract. Similarly, by providing support to the wall of the ventricle, the tethering system may reduce the volume of the left ventricle, making it easier for the heart to pump blood from the left ventricle through the aortic valve.

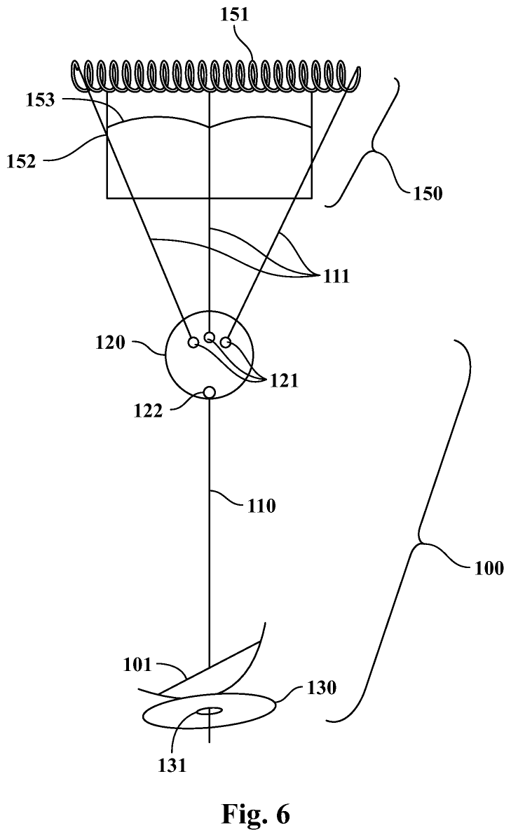

FIG. 6 depicts one example of a tethering system 100 attached to an implanted prosthetic heart valve 150. Although prosthetic heart valve 150 is illustrated as a surgical prosthetic heart valve, the tethering system 100 described herein may also be used in connection with collapsible prosthetic heart valves, with or without modification. For clarity of illustration, the patient anatomy, other than the apex of ventricular wall 101, is omitted from FIG. 6. Prosthetic heart valve 150 may include a sewing cuff 151, valve frame 152, and valve leaflets 153. Sewing cuff 151 may be similar or identical to ring 1200, and may include a covering similar to fabric 1300. As with ring 1200 and/or fabric 1300, sewing cuff 151 may function to surgically secure prosthetic heart valve 150 to a tricuspid or mitral valve along the tricuspid or mitral annulus (not shown) by, for instance, suturing or stitching. Valve frame 152, which may be substantially similar or identical to stent 1100, may be connected to sewing cuff 151 and may assist in providing stability to prosthetic heart valve 150 and providing a structure to support prosthetic heart valve leaflets 153. Valve frame 152 may be partially or fully covered by a covering, such as one similar to fabric 1300. Adjacent valve leaflets 153 may be connected to commissure attachment features of valve frame 152 and may be flexible such that the valve leaflets allow for blood to pass through prosthetic heart valve 150 in an antegrade direction and to substantially block blood from passing through the prosthetic heart valve in a retrograde direction, similar to properly functioning native valve leaflets.

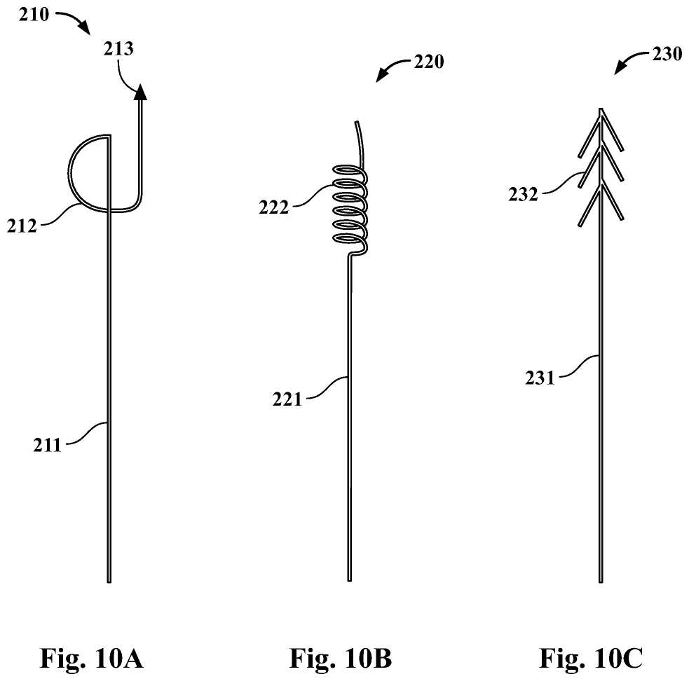

As noted above, tethering system 100 may be provided as a secondary mode of attachment in order to better secure prosthetic heart valve 150 to structures of the heart in the patient, which may reduce stresses on a primary mode of attachment, either to reduce the likelihood of the primary mode of attachment failing, or to otherwise secure the prosthetic heart valve 150 after the primary mode of attachment has already begun to fail. Tethering system 100, which is shown in conjunction with a prosthetic heart valve 150 implanted in native mitral valve 1030, may include valve tethers 111, tether attachment member 120, anchor tether 110, and anchor 130. Valve tethers 111 may be sutures or other thread-like elements, provided singularly or in a braided configuration, and may have a first end attached to sewing cuff 151 and a second opposite end attached to tether attachment member 120. Valve tethers 111 may be attached to sewing cuff 151 in any suitable fashion, including those shown in FIGS. 10A-10C and described below. Although three valve tethers 111 are shown in FIG. 6, it should be understood that more or fewer valve tethers may be used. Preferably, if valve tethers 111 are coupled to sewing ring 151, they are positioned at substantially equal intervals around the circumference of the sewing ring in order to substantially equally distribute forces among the tethers. As described in greater detail below, valve tethers 111 may be attached to prosthetic heart valve 150 and to tether attachment member 120 before surgery, for example during manufacturing of the prosthetic heart valve, during the initial surgical procedure in which the prosthetic heart valve is implanted, or in a subsequent procedure after an initial surgery implanting the prosthetic heart valve.

Tether attachment member 120 may include valve tether attachment features, which may be holes 121, and an anchor tether attachment feature, which may also be hole 122. Valve tether holes 121 may be located on a distal end of tether attachment member 120, and may be designed to secure the proximal end of valve tethers 111 at a central location. Anchor tether hole 122 may be located on a proximal end of tether attachment member 120, and may be designed to engage anchor tether 110 so that the anchor tether is positioned substantially coaxially with prosthetic heart valve 150. In this manner, tether attachment member 120 may serve as a guide for attachment to anchor tether 110 while allowing for a substantially uniform force to be distributed along valve tethers 111 from a tension applied by anchor tether 110. Further, it may be preferable to limit the amount of structures positioned within left ventricle 1024, with the single anchor tether 110 occupying less space than would a plurality of valve tethers 111 extending the entire distance between prosthetic heart valve 151 and anchor 130. In one embodiment, tether attachment member 120 may be a single, integral, laser-cut metallic body as disclosed in U.S. Pat. Pub. No. 2018/0193138, the disclosure of which is incorporated by reference herein.

A proximal end of anchor tether 110 may be connected to anchor 130 through an incision (not shown) in ventricle wall 101. Anchor tether 110 may be secured to anchor 130 at anchor hole 131, for example via a pin that traverses the anchor hole to pierce the anchor tether extending therethrough. Anchor tether 110 may be tightened by the user with a tensioning element (not shown), such as that disclosed in in U.S. Pat. No. 9,986,993, the disclosure of which is incorporated by reference herein. In this manner, a desired tension of anchor tether 110 may be selected, with the amount of tension potentially affecting the proper coaptation of the prosthetic heart valve leaflets 153 and the support provided to left ventricle 1024. Similar to valve tethers 111, anchor tether 110 may be a suture or other thread-like element, either provided singularly or in a braided fashion. In other embodiments, particularly when tethering system is provided in a secondary procedure, valve tethers 111 and anchor tether 110 may be formed of a more rigid material, such as Nitinol or another suitable metal.

Anchor 130 may be positioned on an epicardial surface of the heart along the apex of left ventricle 1024, securing prosthetic heart valve 150 via anchor tether 110, tether attachment member 120, and valve tethers 111. Anchor hole 131 allows for anchor tether 110 to pass through anchor 130 while the anchor is coupled to a tensioning device, so that the anchor tether 110 may be tensioned and secured to the anchor when the anchor is in the desired position at the desired tension. An exemplary anchor is described in greater detail in U.S. Pat. Pub. No. 2016/0143736, the disclosure of which is incorporated by reference herein.

As noted above, tethering system 100 may be coupled to prosthetic heart valve 150 during initial manufacturing of the prosthetic heart valve, just prior to a surgical procedure to implant the prosthetic heart valve, or in a secondary procedure after the prosthetic heart valve has already been implanted into a patient. If tethering system 100 is coupled to prosthetic heart valve 150 during initial manufacturing of the prosthetic heart valve, or just prior to implantation of the prosthetic heart valve, it may be relatively simple to connect valve tethers 111 to prosthetic heart valve 150 and to tether attachment member 120, as well as to connect anchor tether 110 to the tether attachment member, since these attachments may be performed outside the patient's body prior to implantation. In these examples, valve tethers 111 may be coupled to sewing ring 151 in any suitable fashion, for example by tying the valve tethers to the sewing ring, by piercing the sewing ring with the valve tethers, or by using adhesives or other mechanisms. Similarly, both valve tethers 111 and anchor tether 110 may be coupled to tether attachment member 120 in any suitable fashion, including by knotting the tethers to holes within tether attachment member 120, via adhesives, or any other suitable connection mechanism.

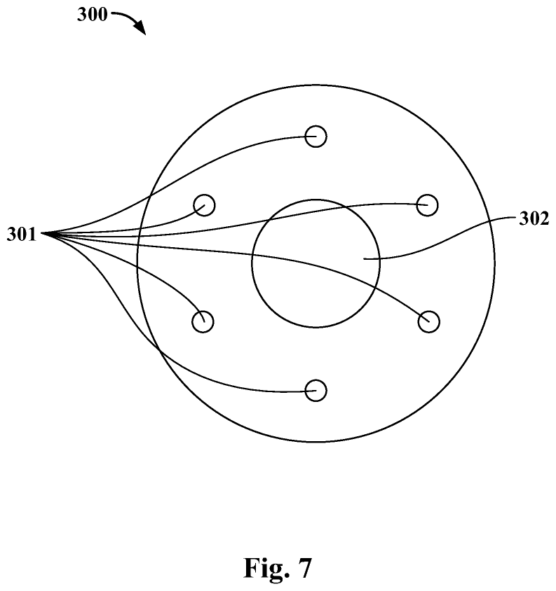

In order to assist attaching valve tethers 111 to prosthetic heart valve 150 at the desired locations, an attachment template may be provided. FIG. 7 depicts an example of a template 300 that includes indicia 301, which may be holes in the template or markings on the template. In the illustrated example, template 300 includes an open interior 302 though which portions of valve stent 152 and leaflets 153 may be passed, so that the indicia 301 of template 300 are positioned in contact with, or in close proximity to, sewing cuff 151. Template 300 may assist in guiding a user to attach valve tethers 111 to sewing cuff 151 at pre-selected locations that correspond to indicia 301. For example, if six valve tethers 111 are to be attached to sewing cuff 151 at equidistance locations around the periphery of the sewing cuff, template 300 may be sized such that, when placed in contact with the sewing cuff, six indicia 301 are positioned at the desired locations of the attachment of valve tethers 111. Valve tethers 111 may then be attached to the desired locations on sewing ring 151, for example by inserting a needle carrying a valve tether through each indicium 301. If indicia 301 are holes, template 300 may be a fabric or rigid material. However, if indicia 301 are merely markings on template 300, the template is preferably formed of a material through which a needle may easily pass, such a fabric. Template 300 may further include features to indicate the desired rotational position of the template relative to prosthetic heart valve 150, particularly if the template is capable of being positioned in more than one rotational position relative to the prosthetic heart valve. Further, it should be understood that template 300 may be best suited for attaching valve tethers 111 to sewing cuff 151 when prosthetic heart valve 150 is being manufactured or otherwise prior to implantation of the prosthetic heart valve. And although template 300 is shown with six indicia 301, it should be understood that any number of indicia corresponding to any desired positioning of valve tethers 111 relative to sewing cuff 151 may be suitable.

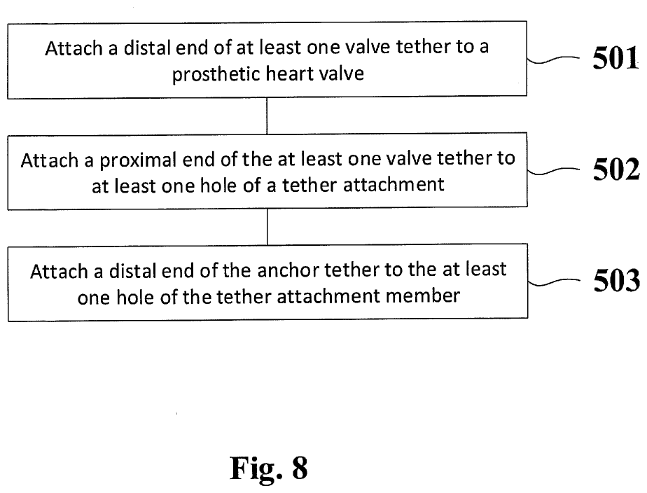

FIG. 8 is a flowchart illustrating a method of implanting prosthetic heart valve 150 into a patient and securing the prosthetic heart valve with tethering system 100, with the tethering system having been attached to the prosthetic heart valve before implantation of the prosthetic heart valve, for example during manufacturing of the prosthetic heart valve or just prior to implantation. At step 501, a distal end of at least one valve tether 111 is attached to the sewing cuff 151 of prosthetic heart valve 150. The one or more valve tethers 111 may be attached using a template similar to template 300, or without the use of such a template. As noted above, each valve tether 111 may be attached to sewing ring 151 via any suitable method, such as knotting. At step 502, a proximal end of the at least one valve tether 111 is attached to tether attachment member 120, for example by tying the valve tether using a hole 121 in tether attachment member 120. At step 503, a distal end of anchor tether 110 is attached to tether attachment member 120, for example via hole 122 in tether attachment member 120. In other words, prior to inserting prosthetic heart valve 150 into the patient's heart, all of the tethers 110, 111 are preferably already coupled to tether attachment member 120. Thus, it should be understood that the order of steps 501, 502, and 503 may not be important.

With the prosthetic heart valve 150 attached to the valve tethers 111 and the anchor tether 110 via the tether attachment member 120, the prosthetic heart valve may be implanted into the native valve annulus, for example via suturing the sewing cuff 151 to the native annulus. This may entail opening the patient's chest and putting the patient on cardio-pulmonary bypass, and making an incision in the left atrium (e.g. an atriotomy, or any standard surgical replacement technique) to gain direct visualization and access to the native mitral valve. Prior to suturing prosthetic heart valve 150 to the native mitral valve annulus, anchor tether 110 is preferably passed through the ventricular wall near the apex or on a free wall of the left ventricle orthogonal to the apex. This may be performed by passing a catheter that surrounds and/or is attached to anchor tether 110 through the ventricular apex from an interior aspect to the exterior aspect. In other embodiments, a catheter may be first passed through the left ventricular apex from the exterior to the interior of the heart, and anchor tether 110 may then be passed through the catheter to traverse the left ventricular apex. With anchor tether 110 traversing the left ventricular apex, prosthetic heart valve 150 may be coupled to the native valve annulus by suturing sewing ring 151 to the annulus tissue. Before or after closing the atriotomy, anchor 130 may be passed over anchor tether 110 and the anchor tether may be tensioned to a desired amount, and the anchor fixed to the anchor tether to maintain the desired tension. Anchor 130 may be coupled to the heart, for example via suturing if desired, and the patient may be taken off bypass to complete the procedure.

It should be understood that, although one illustrative embodiment of prosthetic heart valve 150 and tethering system 100 is described above, various alternative embodiments may provide similarly suitable functionality. For example, although tether attachment member 120 is described above as assisting in centralizing the location of the anchor tether 110 and distributing forces, the tether attachment member is not necessary. For example, each valve tether 111 may extend from a first connection point to prosthetic heart valve 150 to anchor 130, without an intervening tether attachment member. In that circumstance, valve tethers 111 also function as an anchor tether. Omitting tether attachment member 120 may be particularly preferable if valve tethers 111 are being coupled to prosthetic heart valve 150 in a secondary procedure after the prosthetic heart valve has already been implanted into a patient, as the added complexity of attaching valve tethers 111 and an anchor tether 110 to tether attachment member 120 in a transcatheter procedure while the heart is beating may be obviated.

Still further, in some embodiments, valve tethers 111 may be attached to parts of prosthetic heart valve 150 other than (or in addition to) sewing cuff 151, such as valve frame 152. For example, valve frame 152 may be provided with specific features for attachment of valve tethers 111, such as apertures, hooks, or other features. If valve frame 152 is provided with such specific features, a corresponding template 300 may be provided that includes indicia corresponding to those specific features to assist a user in connecting valve tethers 111 to the desired locations. However, it should be understood that such specific features for attachment of valve tethers 111 need not be provided in valve frame 152, and rather the valve tethers may be attached to the valve frame at any suitable locations that allow for such attachment.

Although tethering system 100 is illustrated with three valve tethers 111, it should be understood that the tethering system is not limited to three valve tethers and may comprise any suitable number of valve tethers. Further, valve tethers 111 may be equal or unequal in length to each other. In other embodiments, the anchor tether may be located at an off-angle from the tether attachment.

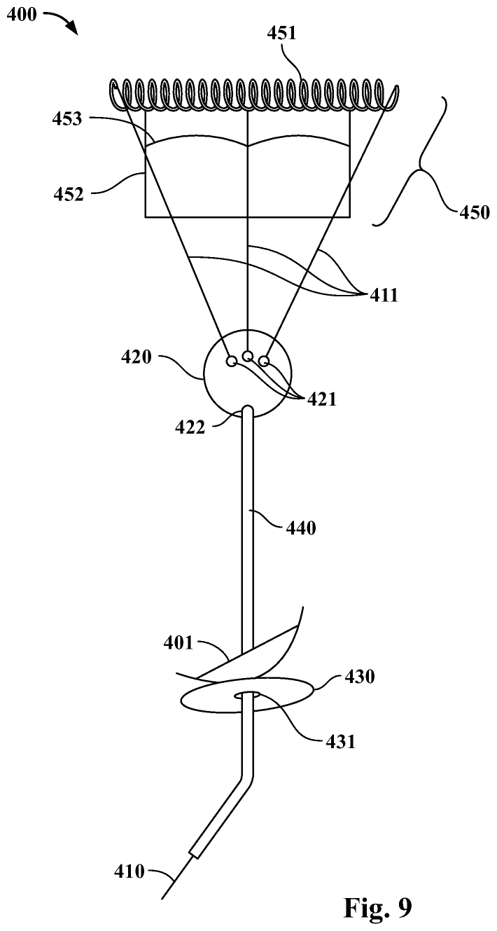

As noted above, while tethering system 100 is described as being coupled to prosthetic heart valve 150 during manufacturing of the prosthetic heart valve or at another time prior to actual implantation of the prosthetic heart valve, similar or identical tethering systems may be coupled, for example during a minimally invasive procedure, to a previously-implanted prosthetic heart valve to provide additional securement of the prosthetic heart valve to the patient's anatomy. For example, FIG. 9 depicts tethering system 400 and prosthetic heart valve 450, which may be similar or identical to tethering system 100 and prosthetic heart valve 150 described above, with certain exceptions such as those noted below. In this embodiment, tethering system 400 is shown in the process of attaching tethering system 400 to prosthetic heart valve 450. In particular, FIG. 9 illustrates three valve tethers 411 having been coupled to sewing cuff 451 of prosthetic heart valve 450, although as noted above, the valve tethers may be additionally or alternately coupled to valve frame 452. Because FIG. 9 illustrates a scenario in which prosthetic heart valve 450 has already been previously implanted into the patient, it should be understood that placing the patient on cardio-pulmonary bypass may not be necessary, and the entire connection of tethering system 400 to the prosthetic heart valve may be performed while the heart remains beating, and the prosthetic leaflets 453 are opening and closing during the normal cardiac cycle. For example, a catheter (not shown) may be introduced into the patient's heart via a transapical puncture of the wall 401 of the left ventricle, with the procedure to connect tethering system 400 to prosthetic heart valve 450 occurring through the catheter. In order to be able to guide valve tethers 411 to the desired connection locations on prosthetic heart valve 450 (which are locations around sewing ring 453 in the example of FIG. 9), it may be preferable for the valve tethers to be formed of a relatively rigid material, for example Nitinol or another suitable biocompatible material, so that the valve tether may be more easily pushed distally through a catheter and toward the connection point at prosthetic heart valve 450.

FIGS. 10A-10C illustrate examples of valve tethers 210, 220, and 230 that may be used for one or more of valve tethers 411 of tethering system 400, although it should be understood that these valve tethers may be suitable for use as one or more valve tethers 111 of tether system 100. In these embodiments, valve tethers 210, 220, 230 each include a respective securing mechanism 212, 222, 232, respectively. FIG. 10A depicts valve tether 210 having tether body 211, securing mechanism 212, and needle tip 213. Securing mechanism 212 may be a knot (e.g. a standard surgeon's knot) formed by passing needle tip 213 through sewing cuff 451 (or through another feature in prosthetic heart valve 450), and tying a knot in tether body 211. Needle tip 213 may take any suitable form, but is preferably sharp to allow for the user to pierce sewing cuff 451 or through other structures of prosthetic heart valve 450. With the distal end of valve tether 210 secured to prosthetic heart valve 450, a proximal end of valve tether 210 may be coupled to tether attachment member 420, or otherwise directly to anchor 430 (for example via anchor hole 431), similar to as described in connection with tethering system 100. Needle tip 213 may be attached to tether body 211 by, for example, crimping, adhesive, or a thermal process. In some instances, it may be desirable for the needle tip 213 to be positioned, following surgery, in a manner so as to avoid causing trauma to surrounding tissue. For example, the needle tip 213 could be positioned within cuff 451 following securement of the valve tether 210. In this embodiment, the needle tip 213 may be formed of a resorbable material so that the needle tip 213 resorbs over time. In other embodiments, the needle tip could be retractable, or a needle transfer technique could be used to remove the needle tip 213 after securement of the valve tether 210.

Although needle tip 213 is illustrated as having a triangular type of shape, in other embodiments, the needle tip may be curved, angled, or have other suitable shapes. Although needle tip 213 may be formed of Nitinol, it should be understood that other materials, including bioabsorbable materials (e.g. poly(lactic acid) or poly(glycolic acid)), may be appropriate.

FIG. 10B depicts valve tether 220 having a tether body 221 and an alternate securing mechanism 222. Securing mechanism 222 may take the form of a coiled shape at a distal end of tether body 221 that can pierce sewing cuff 451 (or another suitable component of prosthetic heart valve 450), and then be rotated about its longitudinal axis to drive the securing mechanism through the sewing cuff.

FIG. 10C depicts valve tether 230 having a tether body 231 and a further alternate securing mechanism 232. Securing mechanism 232 may be one or more barbs, for example arranged in series at a distal end of tether body 231, designed to pierce the sewing cuff 451 of prosthetic heart valve 450 (or other suitable component of the prosthetic heart valve) by advancing the securing mechanism distally. After piercing, the barb(s) may prevent valve tether 230 from being pulled proximally relative to prosthetic heart valve 450, thus securing valve tether 230 to the heart valve. Both valve tethers 220 and 230 may be formed of any of the materials described above in connection with valve tether 210.

If tether attachment member 420 is used with tethering system 400, it may have the same or similar features as described above for tether attachment member 120. In use, it may be preferable for the distal end of each valve tether 411 to be secured to prosthetic heart valve 450, with the proximal end of each valve tether 411 passing through the incision in the wall 401 of the left ventricular apex. The proximal end of each valve tether 411 may be passed through a corresponding hole 421 in tether attachment member 420, which may be positioned outside the heart initially. At that point, tether attachment member 420 may already be coupled to anchor tether 440, for example via hole 422, the anchor tether being similar or identical to anchor tether 110 described above. Tether attachment member 420 may then be slid toward prosthetic heart valve 450, drawing valve tethers 411 inwardly as the tether attachment member 420 travels toward the prosthetic heart valve, and secured in place to valve tethers 411 by a knot. Alternatively, valve tether 411 may be secured to tether attachment member 420 prior to the surgery by a knot or other securement mechanism (e.g. pin, set screw, winding mechanism, or the like), and the length of valve tether 411 may be adjusted at the distal portion of valve tether 411 after being passed through sewing cuff 451 or be predetermined prior to surgery.

With the tether attachment member 420 in the desired position, anchor 431 may be passed over the proximal end of anchor tether 440, and the anchor tether tensioned to a desired amount prior to locking the anchor tether to the anchor, and coupling the anchor to the ventricular apex (for example via sutures) if desired. The proximal end of anchor tether 440 may include a leader 410, which may for example be a Nitinol leader, to assist in positioning the hole 431 in anchor 430 over anchor tether 440, or otherwise to aid in positioning other devices over the proximal end of the anchor tether. For instance, a distal end of leader 410 may be attached to a proximal end of anchor tether 440 by a securing element (not shown), such as a crimp tube or other suitable mechanism.

As with tethering system 100, the use of tethering system 400 may omit tether attachment member 420 and anchor tether 440, with the proximal ends of valve tethers 411 extending through the ventricle wall 401 and secured to the ventricular apex via anchor 430 or a similar anchoring device.

Although tether systems 100 and 400 are described above in relation to a prosthetic mitral valve, it should be understood that similar or identical tethering systems and methods may be used to secure a prosthetic tricuspid valve, with the tethering systems connected to the prosthetic heart valve during manufacturing of the prosthetic heart valve, just prior to implantation, or during a secondary procedure following an initial implantation. Still further, although the description above includes exemplary uses with surgical prosthetic heart valves, as collapsible prosthetic heart valves typically do not include sewing cuffs, it is possible that similar tethering systems may be used in connection with collapsible prosthetic heart valves. In such an instance, the tethering systems in those embodiments would preferably be attached to other features of the collapsible prosthetic heart valve, such as a stent frame.

According to one aspect of the disclosure, a system comprises:

a surgical prosthetic heart valve having a frame, prosthetic leaflets within the frame configured to allow blood to flow through the frame in an antegrade direction but to substantially block blood from flowing through the frame in a retrograde direction, and a cuff configured to be sutured to a native valve annulus of a heart of a patient;

a valve tether having a first end configured to be coupled to the surgical prosthetic heart valve; and

an anchor configured to be secured to a ventricular apex of the heart of the patient; and

wherein, the valve tether includes a second end opposite the first end, the second end configured to be operably coupled to the anchor; and/or

the first end of the valve tether is configured to be attached to the surgical prosthetic heart valve by at least one of a knot, a barb, or a coil; and/or

an attachment member and an anchor tether having a first end and a second end, the attachment member being configured to attach to the second end of the valve tether and to the first end of the anchor tether, the second end of the anchor tether being configured to be secured to the anchor; and/or

the valve tether includes a plurality of valve tethers; and/or

a template having a plurality of indicia, the plurality of indicia corresponding to locations on the surgical prosthetic heart valve intended to couple to the first ends of the plurality of valve tethers; and/or

the first end of the valve tether is configured to attach to the cuff of the surgical prosthetic heart valve; and/or

the first end of the valve tether is configured to attach to the frame of the surgical prosthetic heart valve.

According to another aspect of the disclosure, a system for tethering a prosthetic heart valve to a heart of a patient comprises:

a valve tether having a first end configured to be coupled to the prosthetic heart valve;

an anchor configured to be secured to a ventricular apex of the heart of the patient; and

wherein the valve tether is formed of a rigid material and includes a securing mechanism on the first end to secure the first end of the valve tether to the prosthetic heart valve, the valve tether including a second end opposite the first end configured to be operably coupled to the anchor; and/or

the securing mechanism on the first end of the valve tether is a knot, a barb, or a coil; and/or

the valve tether is formed of a metal; and/or

the metal is a nickel-titanium alloy; and/or

the valve tether includes a plurality of valve tethers; and/or

a template having a plurality of indicia, the plurality of indicia corresponding to locations on the prosthetic heart valve intended to couple to the first ends of the plurality of valve tethers; and/or

an attachment member and an anchor tether having a first end and a second end, the attachment member being configured to attach to the second end of the valve tether and to the first end of the anchor tether, the second end of the anchor tether being configured to be secured to the anchor.

A further aspect of the disclosure is a method of tethering a prosthetic heart valve to a heart of a patient during a second interventional procedure, the prosthetic heart valve having already been implanted into the patient during an earlier first interventional procedure, the method comprising:

inserting the first end of a valve tether into an incision on a ventricular wall of the heart of the patient;

advancing the first end of the valve tether to the previously implanted prosthetic heart valve;

attaching the first end of the valve tether to the prosthetic heart valve;

operably attaching a second end of the valve tether opposite the first end to an anchor; and

securing the anchor to the ventricular wall of the heart of the patient; and/or

coupling the second end of the valve tether to an attachment member, and coupling a second end of an anchor tether to the anchor, a first end of the anchor tether being coupled to the attachment member; and/or

adjusting a tension of the anchor tether prior to securing the second end of the anchor tether to the anchor; and/or

the prosthetic heart valve is a surgical prosthetic heart valve having a frame and a cuff, and attaching the first end of the valve tether to the prosthetic heart valve includes attaching the first end of the at valve tether to at least one of the cuff and the frame of the surgical prosthetic heart valve; and/or

attaching the first end of the valve tether to the prosthetic heart valve includes attaching the first end of the valve tether to the cuff of the surgical prosthetic heart valve by piercing the cuff with a barb, a coil, or a needle tip at the first end of the valve tether; and/or

operably attaching the second end of the valve tether to the anchor includes directly attaching the second of the valve tether to the anchor.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A system comprising:
a surgical prosthetic heart valve having a frame, prosthetic leaflets within the frame configured to allow blood to flow through the frame in an antegrade direction but to substantially block blood from flowing through the frame in a retrograde direction, and a cuff configured to be sutured to a native valve annulus of a heart of a patient;
a valve tether having a first end configured to be coupled to the surgical prosthetic heart valve; and
an anchor configured to be secured to a ventricular apex of the heart of the patient; and
wherein, the valve tether includes a second end opposite the first end, the second end configured to be operably coupled to the anchor.

2. The system of claim 1, wherein the first end of the valve tether is configured to be attached to the surgical prosthetic heart valve by at least one of a knot, a barb, or a coil.

3. The system of claim 1, further comprising an attachment member and an anchor tether having a first end and a second end, the attachment member being configured to attach to the second end of the valve tether and to the first end of the anchor tether, the second end of the anchor tether being configured to be secured to the anchor.

4. The system of claim 1, wherein the valve tether includes a plurality of valve tethers.

5. The system of claim 4, further comprising a template having a plurality of indicia, the plurality of indicia corresponding to locations on the surgical prosthetic heart valve intended to couple to the first ends of the plurality of valve tethers.

6. The system of claim 1, wherein the first end of the valve tether is configured to attach to the cuff of the surgical prosthetic heart valve.

7. The system of claim 1, wherein the first end of the valve tether is configured to attach to the frame of the surgical prosthetic heart valve.

8. A system for tethering a prosthetic heart valve to a heart of a patient, the system comprising:
a valve tether having a first end configured to be coupled to the prosthetic heart valve;
an anchor configured to be secured to a ventricular apex of the heart of the patient; and
wherein the valve tether is formed of a rigid material and includes a securing mechanism on the first end to secure the first end of the valve tether to the prosthetic heart valve, the valve tether including a second end opposite the first end configured to be operably coupled to the anchor.

9. The tethering system of claim 8, wherein the securing mechanism on the first end of the valve tether is a knot, a barb, or a coil.

10. The tethering system of claim 8, wherein the valve tether is formed of a metal.

11. The tethering system of claim 10, wherein the metal is a nickel-titanium alloy.

12. The tethering system of claim 8, wherein the valve tether includes a plurality of valve tethers.

13. The tethering system of claim 12, further comprising a template having a plurality of indicia, the plurality of indicia corresponding to locations on the prosthetic heart valve intended to couple to the first ends of the plurality of valve tethers.

14. The tethering system of claim 8, further comprising an attachment member and an anchor tether having a first end and a second end, the attachment member being configured to attach to the second end of the valve tether and to the first end of the anchor tether, the second end of the anchor tether being configured to be secured to the anchor.

15. A method of tethering a prosthetic heart valve to a heart of a patient during a second interventional procedure, the prosthetic heart valve having already been implanted into the patient during an earlier first interventional procedure, the method comprising:
inserting the first end of a valve tether into an incision on a ventricular wall of the heart of the patient;
advancing the first end of the valve tether to the previously implanted prosthetic heart valve;
attaching the first end of the valve tether to the prosthetic heart valve;
operably attaching a second end of the valve tether opposite the first end to an anchor; and
securing the anchor to the ventricular wall of the heart of the patient.

16. The method of claim 15, further comprising coupling the second end of the valve tether to an attachment member, and coupling a second end of an anchor tether to the anchor, a first end of the anchor tether being coupled to the attachment member.

17. The method of claim 16, further comprising adjusting a tension of the anchor tether prior to securing the second end of the anchor tether to the anchor.

18. The method of claim 15, wherein the prosthetic heart valve is a surgical prosthetic heart valve having a frame and a cuff, and attaching the first end of the valve tether to the prosthetic heart valve includes attaching the first end of the at valve tether to at least one of the cuff and the frame of the surgical prosthetic heart valve.

19. The method of claim 18, wherein attaching the first end of the valve tether to the prosthetic heart valve includes attaching the first end of the valve tether to the cuff of the surgical prosthetic heart valve by piercing the cuff with a barb, a coil, or a needle tip at the first end of the valve tether.

20. The method of claim 15, wherein operably attaching the second end of the valve tether to the anchor includes directly attaching the second of the valve tether to the anchor.
